(19) United States
(12) Patent Application Publication
(10) Pub. No.: US 2021/0138053 A1
(43) Pub. Date: May 13, 2021

(54) ANTI-TUMORAL COMPOSITION

(71) Applicant: Jean-Marc LIMACHER, Matzenheim (FR)

(72) Inventor: Jean-Marc LIMACHER, Matzenheim (FR)

(21) Appl. No.: 17/152,031

(22) Filed: Jan. 19, 2021

Related U.S. Application Data
(63) Continuation of application No. 15/766,510, filed on Apr. 6, 2018.

(30) Foreign Application Priority Data
Oct. 8, 2015 (FR) 15 59589
Sep. 30, 2016 (FR) 16 59450

(51) Int. Cl. A61K 39/00; A61K 9/00; A61K 39/395; C12N 7/00

(52) U.S. Cl. CPC A61K 39/0011; A61K 9/0021; A61K 39/3955; A61K 39/39558; C12N 7/00; A61K 2039/53; A61K 2039/54; C12N 2710/24143; A61K 2039/505; A61K 2039/55516; A61K 2039/5252; A61K 2039/5254; C12N 2710/24132

(57) ABSTRACT

A pharmaceutical composition characterised in that it comprises at least one poxvirus, a naked nucleic acid sequence encoding at least one specific neoantigen of a cancerous cell and an anti-CTLA4 antibody.

### CROSS-REFERENCE TO RELATED APPLICATIONS

This is a Continuation application of a non-provisional application having U.S. patent application Ser. No. 15/766,510 entitled “ANTI-TUMORAL COMPOSITION” filed on Apr. 6, 2018, and the National Phase application of International Application No. PCT/FR2016/052598 filed on Oct. 7, 2016, which claims priority to French Application No. 16 59450 filed on Sep. 30, 2016, and French Application No. 15 59589 filed on Oct. 8, 2015, the contents of which are all incorporated herein by reference.

### TECHNICAL FIELD

The present invention relates to a pharmaceutical composition characterised in that it comprises at least one nucleic acid sequence encoding at least one specific neoantigen of a tumour, a poxvirus, and an anti-CTLA4 antibody. The present invention also relates to the use of compositions according to the invention for the treatment of cancer.

### PRIOR ART

Traditionally, in the context of tumour diseases, the treatments consist of the use of chemical or biological compounds (chemotherapy), radiation (radiotherapy) and/or surgery. Even though tremendous progress has been observed in recent years with targeted therapies particularly using monoclonal antibodies or other molecules targeting specific receptors, high mortality is still observed, and further therapeutic pathways continue to be envisaged in order to lower the associated mortality and/or side-effects associated with these diseases and with the treatments thereof.

Among the new therapeutic pathways envisaged, particular mention may be made of gene therapy and immunotherapy.

The idea of immunising against pre-existing cancer cells is based on the observation that cancer cells express mutated proteins, abnormally glycosylated proteins or, in the case of tumours induced by viral infection, viral proteins. Within the tumour, the expression of these proteins does not induce the production of an effective immune response. Indeed, the tumour is particularly capable of creating a microenvironment preventing the initiation of an immune response targeting these antigens and/or the cells expressing same. Furthermore, even if an immune response is induced, it does not always succeed in being effective due to the anergy prevailing in the tumour. It has thus been postulated that it is more effective, in terms of immune response generated to inject the antigens associated with the tumour cells into a zone where the immune system is active.

Among the antigens associated with tumours, mention may also be made of tumour neoantigens obtained from the accumulation of mutations in a cell population giving rise to cancerisation. These mutations may result for example from exposure to ultraviolet radiation or carcinogenic substances from tobacco or of other kinds. Some are directly involved in carcinogenesis, others are merely in a “passenger” situation but all, due to the change of amino acids in the sequence of a protein, have the potential to be considered by the immune system as different from the self and therefore antigenic.

In the context of cancer treatment, gene therapy has been used according to three main areas. The first consisted of injecting on the tumour site genes encoding immunostimulant molecules suitable for lifting the local inhibition of the immune system. For example, genes encoding interleukins were injected directly into tumours. Local interleukin production was supposed to enable the normal activity of the immune system and the destruction of cancer cells. However, the results obtained with this type of strategy have been disappointing.

A second area of research consists of injecting into cancer cells genes encoding cytotoxic molecules or inducing the direct or indirect production of cytotoxic molecules. This strategy is supposed to enable the destruction of tumour cells and optionally the release of antigens into the body thereby enabling the activation of the immune system.

The final area of research consisted of using nucleic acids encoding antigens associated with tumours to vaccinate patients. Indeed, the production of the antigen by the patient's cells, rather than the in-situ injection thereof, makes it possible to envisage superior antigen presentation more in keeping with that occurring in cancer cells.

However, the antitumoral response observed in the context of these various treatments is not much better than the clinical responses observed in the context of conventional treatments.

It is hence desirable to have novel products and/or novel methods enabling long-term control of the tumour volume and an increase in the survival rate of patients treated.

### SUMMARY OF THE INVENTION

The present invention relates to a pharmaceutical composition characterised in that it comprises at least one nucleic acid sequence encoding at least one specific neoantigen of a cancer cell, at least one poxvirus, and an anti-CTLA4 antibody.

The specific combination of these three components makes it possible to obtain an immune response targeted against the neoantigen(s) encoded by the nucleic acid sequence. The poxvirus makes it possible both to enhance the transfection of the nucleic acid sequence and enhance the quality of the immune response generated against the neoantigen(s). The anti-CTLA-4 antibody also makes it possible to enhance the immune response against the neoantigen(s). The use of a nucleic acid sequence makes it possible to envisage rapid and personalised production of the sequence encoding the neoantigen(s). Indeed, current techniques enable the full sequencing of the genotype of a patient's cancer cells and identify specific mutations by constitutional comparison of the patient. If these mutations are non-synonymous, belong to coding sequences expressed by the tumour, and are due to the characteristics thereof supposed to be immunogenic, then the latter may be used to induce immunisation against the tumour.

The mutations in the genome of the tumours and the resulting neoantigens are a unique combination restricted to the tumour of a given patient, or to the clones forming this tumour.

The use of neoantigens as a means for immunising a patient against his/her own tumour cells is a novel concept. The implementation thereof is rendered complex by the need to produce in a short time a personalised pharmaceutical preparation corresponding to the neoantigens carried by the tumour of a given patient.

To date, therapeutic immunisation in oncology has made use of two major categories of products. The first consists of administering, generally by injection, peptides or proteins supposed to be antigens present in the tumour, the whole in the presence of adjuvant substances the use whereof sometimes raises concerns. The second consists of administering nucleic acids encoding antigens supposed to be present in the tumour but generally corresponding to proteins expressed preferentially by the tumour but which are not necessarily different from the non-tumoral version and do not necessarily comply with the definition of a neoantigen.

Of these two product classes, immunotherapies using gene therapy, particularly those based on the use of viral vectors, have demonstrated reproducibly significant efficacy in the treatment of tumours Immunotherapies using gene therapy, above all those with a viral base, are naturally perceived by the immune system as a danger signal. They are more immunogenic and do not require in principle the use of adjuvants. The viral component, particularly if it consists of a poxvirus, induces an innate response promoting the development of an adaptive immune response against the antigens expressed.

The design and pharmaceutical production of a genetically modified virus expressing the sequences encoding one or a plurality of neoantigens present in a patient's tumour is a technically feasible option; however, it involves a long and costly operation that is very difficult to generalise.

Conversely, the production of plasmids, or of double-stranded closed linear nucleic acids, or of other genetic expression vectors encoding one or a plurality of neoantigens is a simple, relatively inexpensive operation, feasible in a few weeks at most.

The principle of co-injecting a nucleic acid encoding one or a plurality of neoantigens and of a virus, in particular a poxvirus, is liable to enable the production in a short time of a personalised pharmaceutical preparation corresponding to the neoantigens carried by a patient's tumour while benefiting from the presence of a virus supplying the danger signal necessary for the development of the immune response.

Furthermore, the poxvirus, due to the phospholipidic components of the membrane thereof, is liable to promote the penetration of the nucleic acid into the cells present in the vicinity of the injection site, and hence the expression of the antigenic proteins encoded by the nucleic acid.

The nucleic acid sequence in the case of a DNA expression vector promotes the recognition by specific sensors of the double-stranded DNA in the cytoplasm of the cells wherein these vectors have penetrated. This recognition is the source of a biological response promoting the immune response.

The nucleic acid sequence and the poxvirus therefore have different action pathways which are complementary and make it possible to obtain a superior response.

A further advantage of the poxvirus lies in the ability thereof to optionally express additional tumour antigens which may be expressed and presented at the injection site in addition to the neoantigens. By way of example, mention may be made of the HPV E6 and E7 proteins in the case of the tumour induced by this virus, or the MUC1 protein in the case of adenocarcinomas.

Anti-CTLA4 antibodies are inhibitors of a negative checkpoint of the immune response. They were hitherto administered by the systemic, intravenous route, with significant efficacy, at least in the treatment of malignant skin melanoma and non-small cell lung cancer, but in exchange for non-negligible adverse effects.

The co-administration of an anti-CTLA4 antibody or antibody fragment is liable to boost the immune response induced by immunotherapy, in particular the combination of a genetic expression vector and a poxvirus. For this, doses reduced to ½^{th} or less of the usual systemic dose are supposed to be sufficient. At these doses and after co-injection, the concentration in the drainage lymph node of the injection site is supposed to be higher than via the systemic route whereas the systemic concentration per se is greatly reduced, the risk of adverse effects likewise.

Within the scope of the present invention, the term “nucleic acid sequence” refers to a DNA or RNA type coding nucleic acid sequence. This nucleic acid sequence is not included in a viral genome or encapsulated in a viral particle.

According to one embodiment of the invention, said nucleic acid sequence is selected from the group comprising plasmids and vectors of linear DNA or RNA.

According to one embodiment of the invention, said nucleic acid sequence is naked, i.e. it is not associated with molecules promoting the penetration thereof in eukaryotic cells such as, for example, cationic polymers, viral polymers, lipids and/or liposomes.

According to a preferred embodiment of the invention, said nucleic acid sequence is a double-stranded closed linear nucleic acid. Furthermore, closed linear nucleic acids due to the production method thereof are accompanied by very little production residue, in particular bacterial, which makes it possible to avoid burdensome purification steps and enables quicker and less costly use.

Closed linear nucleic acids are well-known to those skilled in the art, the structure thereof, the functions thereof and the processes enabling the production thereof are particularly described in the documents CN103080337, EP2601312, GB201013153, JP2013535210, US2013216562, WO12017210, AU2010209532, CA2751130, CN102301010, DK2391731, EA021069, EA201101141, EP2391731, EP2612925, ES2400890, GB200901593, HK1159693, IL213930, IN05006CN2011, JP2012516147, KR20110107846, MX2011007937, NZ594004, SG173102, US2012282283, U.S. Pat. No. 9,109,250, W010086626.

Within the scope of the present invention, the term “neoantigen associated with a cancer cell” refers to a protein expressed by the cancer cell in a mutated form by comparing with the patient's constitutional sequence.

According to a preferred embodiment of the invention, the anti-CTLA-4 antibody is preferentially chosen from the group comprising ipilimumab and tremelimumab. It may also consist of an antibody fragment targeted against CTLA-4 or any molecule containing a paratope specifically targeted against CLTA4. For the purposes of clarity, it is specified that the term “anti-CTLA4 antibody” does not apply to nucleic acids encoding an anti-CLTA4 antibody.

According to a preferred embodiment, said nucleic acid sequence further comprises regulatory elements providing the expression of the specific neoantigens of a cancer cell in eukaryotic cells.

According to a preferred embodiment, the regulatory elements providing the expression of the neoantigen(s) associated with a cancer cell in the eukaryotic cells include a gene transcription promoter and a translation initiation region in the host cells. Said nucleic acid sequence may comprise a plurality of sequences each encoding a different neoantigen associated with the tumours. In the case of a plurality of neoantigens, the sequences encoding the latter may be placed under the dependency of identical or different regulatory elements.

According to a preferred embodiment, the poxvirus is live or killed.

According to a preferred embodiment, the poxvirus does not comprise heterologous sequences.

According to a preferred embodiment, the poxvirus derives from a vaccinia virus, a canarypox or a fowlpox.

According to a preferred embodiment, said poxvirus derives from a vaccinia virus selected from the Copenhagen, Wyeth and Modified Ankara (MVA) strains.

According to a preferred embodiment, said poxvirus derives from a Copenhagen strain vaccinia virus. According to a preferred embodiment, said poxvirus derives from an MVA strain vaccinia virus.

Within the scope of the present invention, the term “derives” infers that said virus belongs to said strain.

According to a further preferred embodiment, said poxvirus includes a heterologous DNA sequence encoding at least the essential region of an antigen associated with a cancer cell.

According to a preferred embodiment, the DNA sequences encoding at least the essential region of an antigen associated with a cancer cell are under the control of a poxvirus gene promoter.

According to a preferred embodiment, the sequences encoding at least the essential region of an antigen associated with a cancer cell are placed under the control of a vaccinia virus gene promoter and, in particular of a promoter selected from the thymidine kinase (TK), 7.5K, H5R and K1 L gene promoters.

According to an even more preferred embodiment, said promoter is the promoter of the vaccinia virus 7.5 K protein gene.

According to a preferred embodiment, the DNA sequences encoding at least the essential region of an antigen associated with a cancer cell are inserted inside a non-essential region of the virus used.

According to a preferred embodiment, the non-essential region is the TK gene.

In the case where said poxvirus derives from a Copenhagen strain vaccinia virus, the sequences encoding at least the essential region of an antigen associated with a cancer cell are preferentially inserted at the TK and/or KIL locus of said vaccinia virus.

In the case where said poxvirus derives from an MVA strain vaccinia virus, the sequences encoding at least the essential region of an antigen associated with a cancer cell are preferentially inserted at one at least of excision zones I to VI of said vaccinia virus and particularly II and/or III.

The present invention also relates to a composition according to the invention further comprises a pharmaceutically acceptable substrate enabling the administration thereof by injection to humans or animals.

According to a preferred embodiment of the invention, said pharmaceutically acceptable substrate enables the injection of said composition via a needleless injection device.

Within the scope of the present invention, the term “needleless injection device” refers to a device for intradermal, subcutaneous or intramuscular injections, of a liquid active substance for therapeutic use in human or veterinary medicine using a pressurised liquid stream to penetrate the tissues. The liquid may be more or less viscous, a liquid mixture, or a gel.

The administration of nucleic acids generally requires the use of an electroporation technique coupled with injection. Pressurised injection via a needleless system is a further solution. It also has the advantage due to the pressurised injection of ensuring superior diffusion in the tissue spaces. The use thereof also enables if required the extemporaneous preparation of a mixture of products to be injected. The use of a needleless pressurised injection system is liable to promote superior tissue penetration than with a conventional needle and therefore the transfection of a greater number of cells. The aim is to thereby promote the immune response against the neoantigens expressed by the nucleic acid sequence.

The present invention also relates to a set of parts comprising a nucleic acid sequence encoding at least one specific neoantigen of a cancer cell, at least one poxvirus, and an antibody or antibody fragment targeted against CTLA4.

Said set of parts may particularly be used for preparing extemporaneously a composition according to the invention.

The present invention also relates to the use of a composition according to the invention for the treatment of a cancer or a tumour.

The present invention also relates to the use of a composition according to the invention for the treatment of a cancer of the cervix uteri, the ENT sphere or any other tumour induced by the HPV virus, of a liver cancer associated with chronic hepatitis B or C virus, or of a cancer expressing MUC1 protein.

### DESCRIPTION OF EMBODIMENTS

According to a preferred embodiment of the invention, the cancer cells of a patient to be treated are analysed using a biopsy or an operative specimen. The DNA and RNA of the cancer cells, as well as the constitutional DNA obtained from a blood sample, are extracted and sequenced.

The DNA sequences of the healthy cells is compared to the DNA sequence of the cancer cells and the somatic mutations comprised in coding and expressed regions are identified.

The coding and expressed mutated sequences are processed using algorithms suitable for defining for example the most antigenic and may serve as a target. These expressed, coding, non-synonymous, mutated sequences, specific of the patient's cancer cells, referred to as neoantigens, are subsequently generated by DNA or DNA synthesis and then placed in the form of an expression vector (plasmid or closed linear nucleic acid or RNA). According to a preferred embodiment of the invention, using the method described in the patent application EP2391731, the nucleic acid sequence encoding one or a plurality of neoantigens associated with the patient's tumour is placed, inside the closed linear nucleic acid, under the dependency of the sequences required for the expression thereof in eukaryotic cells. As such, said sequence is preferentially placed downstream from a eukaryotic expression promoter and upstream from a transcription termination sequence. It may consist of a strong eukaryotic promoter and in particular a CMV early promoter. The promoter is either of viral origin, or of cellular origin. By way of viral promoter other than CMV, mention may be made of the early or late promoter of SV40 virus or the LTR promoter of Rous sarcoma virus. By way of cellular promoter, mention may be made of the promoter of a cytoskeleton gene, such as for example the desmin promoter, or the actin promoter. Preferentially, an intron may be integrated inside said sequence encoding one or a plurality of neoantigens associated with the patient's tumour. Indeed, some introns are known to increase the transcription of nucleic acid sequences.

The poxvirus used in the composition is preferentially an MVA virus. The latter derives from the Ankara strain of the vaccinia virus by successive passages on chick embryo cells. These different passages induced the attenuation of this virus which could thereby be used in the latest vaccination campaigns against smallpox.

The description of the different MVA strains, the methods suitable for optionally inserting any exogenous genes in the genome thereof as well as the methods of production, and of purification of this virus are particularly accessible in the documents WO0168820, WO0242480, W003008533, W003048184, W003053463, W003054175, WI03088994, WO03097675, WO03097844, WO03097845, WO03097846, WO04048582, WO04048606, WO05054484, WO06089690, WO08028665, WO08045346, WO08131926, WO08131927, WO08138533, WO09052328, WO9152969, WO10057650, WO10060632, WO10102822, WO11042180, WO11092029, WO12010280, WO12048817, WO12059243, WO13083254, WO13189611, WO14019718, WO14037124, WO14062778, WO14063832, WO9813500, WO9915692.

The poxvirus may also comprise sequences encoding further specific antigens of the tumour affecting the patient. For example, the sequences encoding HPV E6 and E7 antigens or the sequences encoding HCV NS3, NS4, NS5B antigens, or the Muc1 protein abnormally glycosylated in the tumours.

Finally, the composition according to the invention will preferentially comprise an anti-CTLA4 antibody or antibody fragment, even more preferentially ipilimumab. Within the scope of the present invention, the term antibody also denotes the bispecific antibodies comprising at least one specific paratope of CTLA-4.

Preferably, the composition according to the invention further comprises a pharmaceutically acceptable substrate. Within the scope of the present invention, the “pharmaceutically acceptable substrate” denotes all substrates, solvents, diluents, excipients, adjuvants, dispersion media, and equivalent, compatible with pharmaceutical administration.

The composition to be used in the invention is suitably buffered so as to be suitable for human use at a physiological or slightly alkaline pH.

The composition according to the invention may be administered to the patient by a variety of methods of administration such as for example the subcutaneous, intradermal, intramuscular, intravenous, intraperitoneal, intratumoral, intravascular, intra-arterial routes.

The injections may be made with conventional syringes and needles, but preferentially via a bioject(r) type needleless injection device.

The administration of the composition according to the invention may take place as a single or repeated dose after a certain time interval ranging from one day to one year. Preferentially, the administration will take place weekly seven times in succession and then subsequently once every three weeks.

The suitable dosage may be adapted according to various parameters, in particular the method of administration, the composition used, the age, health, and weight of the host body, the nature and extent of the symptoms, the associated treatment type, the treatment frequency.

Those skilled in the art are capable of determining the suitable quantities of each element within the composition according to the invention. By way of example, the poxvirus may be used at a quantity between 10⁴ to 10⁹ pfu, the naked nucleic acid sequence in a quantity between 10 μg and 20 mg, and the anti-CTLA4 antibody in a quantity between 10 ng and 20 mg per injection.

Preferentially, the composition according to the invention may be used in conjunction with radiotherapy, chemotherapy, surgery and/or further immunotherapy products such as anti-PD1 and anti-PDL1 antibodies, or the combination of a plurality of these treatments at once.

1. A method of treating a cancer or a tumor in a subject, comprising:
administering by subcutaneous injection to said subject a therapeutically effective amount of a composition comprising at least one nucleic acid sequence encoding at least one specific neoantigen of a cancer cell or a tumor cell, at least one poxvirus, and an anti-CTLA4 antibody, wherein the at least one nucleic acid sequence is not included in a viral genome or encapsulated in a viral particle.

2. The method according to claim 1, wherein said at least one nucleic acid sequence further comprises regulatory elements providing the expression of the specific neoantigens of a cancer cell in eukaryotic cells.

3. The method according to claim 2, wherein the regulatory elements providing the expression of the neoantigens associated with said cancer cell in the eukaryotic cells include a gene transcription promoter and a translation initiation region in the host cells.

4. The method according to claim 1, wherein said at least one nucleic acid sequence is selected from the group consisting of plasmids, closed linear DNA vectors, and RNA molecules.

5. The method according to claim 1, wherein said at least one nucleic acid sequence is a closed linear nucleic acid sequence.

6. The method according to claim 1, wherein said at least one poxvirus does not comprise heterologous sequences.

7. The method according to claim 1, wherein said at least one poxvirus is live or killed.

8. The method according to claim 1, wherein said at least one poxvirus derives from a vaccinia virus, a canarypox or a fowlpox.

9. The method according to claim 1, wherein the composition further comprises a pharmaceutically acceptable substrate enabling the administration thereof by injection to the subject.

10. The method according to claim 1, wherein said administration by subcutaneous injection is performed using a syringe or a needle.

11. The method according to claim 1, wherein said administration by subcutaneous injection is a pressurized injection via needleless injection device.

12. The method according to claim 11, wherein said pharmaceutically acceptable substrate enables the injection of said composition via a needleless injection device.

13. The method according to claim 1, wherein said administration by subcutaneous injection of said composition is performed as a single dose or repeated dose after a set time interval ranging from one day to one year.

14. The method according to claim 1, wherein said composition comprises from 104 to 109 pfu of said poxvirus, between 10 μg and 20 mg of said at least one nucleic acid sequence, and between 10 ng and 20 mg of said anti-CTLA4 antibody.

15. The method according to claim 1, further comprising treating said subject with at least one of radiotherapy, chemotherapy, and/or immunotherapy products in conjunction with said administration by subcutaneous injection or in combination with said administration by subcutaneous injection at once.

16. The method according to claim 1, wherein said cancer is a cancer of cerix uteri, a liver cancer associated with chronic hepatitis B or C virus, or a cancer expressing MUC1 protein.

17. The method according to claim 1, wherein said tumor is a tumor induced by HPV virus.
